(19) United States
(12) Patent Application Publication
(10) Pub. No.: US 2024/0081914 A1
(43) Pub. Date: Mar. 14, 2024

(54) PATIENT-SPECIFIC SURGICAL METHODS AND INSTRUMENTATION

(71) Applicant: Treace Medical Concepts, Inc., Ponte Vedra, FL (US)

(72) Inventors: Adam D. PERLER, St. Petersburg, FL (US); James Q. SPITLER, Winter Garden, FL (US)

(21) Appl. No.: 18/517,269

(22) Filed: Nov. 22, 2023

Related U.S. Application Data
(62) Division of application No. 17/020,630, filed on Sep. 14, 2020.
(60) Provisional application No. 62/900,294, filed on Sep. 13, 2019.

(51) Int. Cl. A61B 34/10; A61B 17/15; A61B 17/17; G06F 30/10; G06T 7/00; G06T 11/00; G16H 20/40; G16H 30/20; G16H 30/40; G16H 50/50

(52) U.S. Cl. CPC A61B 34/10; A61B 17/152; A61B 17/1775; G06F 30/10; G06T 7/0012; G06T 11/008; G16H 20/40; G16H 30/20; G16H 30/40; G16H 50/50; A61B 2017/565; A61B 2034/108; G06T 2207/30008

(57) ABSTRACT

A method may be used to correct a condition present in a patient. The method may include obtaining a first bone model of a first bone of one or more bones of the patient's foot and using at least the first bone model to generate a cutting guide model. The cutting guide model may define a first bone engagement surface shaped to match a first contour on the first bone, and a first guide feature that, with the first bone engagement surface overlying the first contour, is positioned to guide resection of the one or more bones as part of a surgical osteotomy for correcting the condition. The surgical procedure may be selected from a first group consisting of a bunion correction osteotomy, an Evans calcaneal osteotomy, and a medializing calcaneal osteotomy. The first bone may be selected from a second group consisting of a metatarsus, a cuneiform, and a calcaneus.

### CROSS-REFERENCE TO RELATED APPLICATIONS

The present application is a divisional of U.S. patent application Ser. No. 17/020,630, filed on Sep. 14, 2020, entitled PATIENT-SPECIFIC SURGICAL METHODS AND INSTRUMENTATION, which claims the benefit of U.S. Provisional Patent Application Ser. No. 62/900,294, entitled PATIENT-SPECIFIC SURGICAL METHODS AND INSTRUMENTATION, which was filed on Sep. 13, 2019. The above-referenced applications are incorporated by reference herein as though set forth in their entirety.

### TECHNICAL FIELD

The present disclosure relates to surgical devices, systems, instruments, and methods. More specifically, the present disclosure relates to patient-specific cutting guides and implants, and methods of designing and using the same.

### BACKGROUND

Various bone conditions may be corrected through the use of an osteotomy, in which one or more bones are cut, replaced, and/or reoriented. Cutting guides are often used to help the surgeon properly locate the cut. Unfortunately, many known cutting guides are not patient-specific, and can be difficult to properly position to perform the osteotomy on a specific patient. Even if properly positioned, many known cutting guides are difficult to secure at the desired position, without moving away from the desired position prior to performance of the osteotomy. As a result, many known osteotomy procedures carry risk of an improper cut that fails to correct the underlying condition, or even endangers surrounding tissues.

### SUMMARY

The various systems and methods of the present disclosure have been developed in response to the present state of the art, and in particular, in response to the problems and needs in the art that have not yet been fully solved by currently available osteotomy systems and methods.

In some embodiments, a method may be used to correct a condition present in a patient. The method may include obtaining a first bone model of a first bone of one or more bones of the patient's foot, and using at least the first bone model to generate a cutting guide model. The cutting guide model may define a first bone engagement surface shaped to match a first contour on the first bone, and a first guide feature that, with the first bone engagement surface overlying the first contour, is positioned to guide resection of the one or more bones as part of a surgical osteotomy for correcting the condition. The surgical procedure may be selected from a first group consisting of a bunion correction osteotomy, an Evans calcaneal osteotomy, and a medializing calcaneal osteotomy. The first bone may be selected from a second group consisting a metatarsus, a cuneiform, and a calcaneus.

The one or more bones may include the cuneiform and the metatarsus. The surgical osteotomy may be the bunion correction osteotomy. The condition may be a bunion, and the first guide feature may be positioned to guide resection of one of the cuneiform and the metatarsus.

The first bone may be the cuneiform. The first guide feature may be positioned to guide resection of the cuneiform. The cutting guide model may further define a second bone engagement surface shaped to match a second contour of the metatarsus, and a second guide feature that, with the second bone engagement surface overlying the second contour, is positioned to guide resection of the metatarsus.

The method may further include obtaining a second bone model of the metatarsus, and virtually repositioning the second bone model relative to the first bone model to simulate reorientation of the metatarsus relative to the cuneiform to correct the bunion.

The cutting guide model may further include a first end having the first bone engagement surface, a second end having the second bone engagement surface, a first bone attachment feature positioned to secure the first end to the cuneiform, and a second bone attachment feature positioned to secure the second end to the metatarsus.

The method may further include using the cutting guide model to fabricate a cutting guide having the first bone engagement surface, the second bone engagement surface, the first bone attachment feature, the second bone attachment feature, the first guide feature, and the second guide feature.

The method may further include placing the cutting guide such that the first bone engagement surface overlies the first contour and the second bone engagement surface overlies the second contour, securing the first bone attachment feature to the cuneiform, securing the second bone attachment feature to the metatarsus, using the first guide feature to guide motion of a cutter to resect the cuneiform, and using the second guide feature to guide motion of a cutter to resect the metatarsus.

The method may further include reorienting the metatarsus relative to the cuneiform and, after reorienting the metatarsus relative to the cuneiform, promoting fusion between the cuneiform and the metatarsus.

Obtaining the first bone model may include obtaining CT scan data of the first bone. Using the first bone model to generate the cutting guide model may include converting the CT scan data to a CAD models, using the CAD model to obtain the first contour, and using the first contour to generate the first bone engagement surface of the cutting guide model.

The surgical osteotomy may be the Evans calcaneal osteotomy. The first bone may be the calcaneus. The cutting guide model may further have a second bone engagement surface shaped to match a second contour of the calcaneus such that, with the first bone engagement surface overlying the first contour and the second bone engagement surface overlying the second contour, the first guide feature is positioned to guide a cutter to resect the calcaneus to perform the Evans calcaneal osteotomy.

The surgical osteotomy may be the medializing calcaneal osteotomy. The first bone may be the calcaneus. The cutting guide model may further include a second bone engagement surface shaped to match a second contour of the calcaneus such that, with the first bone engagement surface overlying the first contour and the second bone engagement surface overlying the second contour, the first guide feature is positioned to guide a cutter to resect the calcaneus to perform the medializing calcaneal osteotomy.

The method may further include using at least the first bone model to generate an implant model defining a first bone-facing surface with a first shape that matches a first profile of a first resected surface of the first bone after resection of the first bone with a cutting guide fabricated using the cutting guide model.

The implant model may further have a second bone-facing surface with a second shape that matches a second profile of a second resected surface of the first bone or a second bone of the one or more bones after resection of the first bone or a second bone with the cutting guide.

The method may further include using the cutting guide model to fabricate a cutting guide having the first bone engagement surface and first guide feature, using the implant model to fabricate an implant having the first bone-facing surface and the second bone-facing surface, placing the cutting guide such that the first bone engagement surface overlies the first contour, using at least the first guide feature to guide motion of a cutter to resect the one or more bones to define the first resected surface and the second resected surface, and placing the implant between the first resected surface and the second resected surface such that the first shape is aligned with the first profile and the second shape is aligned with the second profile.

According to one embodiment, a system may be provided for correcting a condition present in one or more bones of a patient's foot. The system may have a cutting guide with a first bone engagement surface shaped to match a first contour on a first bone of the one or more bones, and a first guide feature that, with the first bone engagement surface overlying the first contour, is positioned to guide resection of the one or more bones as part of a surgical osteotomy for correcting the condition. The surgical osteotomy may be selected from a first group consisting of a bunion correction osteotomy, an Evans calcaneal osteotomy, and a medializing calcaneal osteotomy. The first bone may be selected from a second group consisting of a metatarsus, a cuneiform, and a calcaneus.

The first bone may be the cuneiform. The surgical osteotomy may be the bunion correction osteotomy. The condition may be a bunion. The first guide feature may be positioned to guide resection of the cuneiform. The cutting guide may further have a second bone engagement surface shaped to match a second contour of the metatarsus, and a second guide feature that, with the second bone engagement surface overlying the second contour, is positioned to guide resection of the metatarsus.

The cutting guide may further have a first end having the first bone engagement surface, a second end having the second bone engagement surface, a first bone attachment feature positioned to secure the first end to the cuneiform, and a second bone attachment feature positioned to secure the second end to the metatarsus.

The surgical osteotomy may be the Evans calcaneal osteotomy or the medializing calcaneal osteotomy. The first bone may be the calcaneus. The cutting guide may further have a second bone engagement surface shaped to match a second contour of the calcaneus such that, with the first bone engagement surface overlying the first contour and the second bone engagement surface overlying the second contour, the first guide feature is positioned to guide a cutter to resect the calcaneus to perform the Evans calcaneal osteotomy or the medializing calcaneal osteotomy.

The system may further have an implant with a first bone-facing surface with a first shape that matches a first profile of a first resected surface of the first bone after resection of the first bone with the cutting guide, and a second bone-facing surface comprising a second shape that matches a second profile of a second resected surface of the first bone or a second bone of the one or more bones after resection of the first bone or a second bone with the cutting guide.

According to some embodiments, a cutting guide may be provided for correcting a bunion present a patient's foot. The cutting guide may have a first bone engagement surface shaped to match a first contour on cuneiform of the patient's foot, and a second bone engagement surface shaped to match a second contour on a metatarsus of the patient's foot. The cutting guide may further have a first slot that, with the first bone engagement surface overlying the first contour and the second bone engagement surface overlying the second contour, is positioned to guide resection of the cuneiform to define a first resected surface on the cuneiform. Further, the cutting guide may have a second slot that, with the first bone engagement surface overlying the first contour and the second bone engagement surface overlying the second contour, is positioned to guide resection of the metatarsus to define a second resected surface on the metatarsus. The first slot and the second slot may be positioned and oriented relative to each other such that, upon fusion of the cuneiform and the metatarsus between the first resected surface and the second resected surface, the bunion is at least partially corrected.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings in which:

FIG. 1A is a flowchart diagram depicting a method for correcting a bone condition, according to one embodiment.

FIG. 1B is a flowchart diagram depicting a method for correcting bunion deformity of the human foot, according to one embodiment.

FIG. 2 is a perspective view of a portion of a foot with a bunion deformity to be treated through use of the methods of FIGS. 1A and/or 1B, according to one embodiment.

FIGS. 3A, 3B, 3C, and 3D are top perspective, alternative top perspective, front elevation, and bottom perspective views, respectively, of a patient-specific cutting guide, according to one embodiment.

FIG. 4 is a perspective view of the foot of FIG. 2, with the cutting guide of FIGS. 3A, 3B, 3C and 3D properly positioned on the first cuneiform and the first metatarsus, but as yet not attached to the first cuneiform and the first metatarsus.

FIG. 5 is a perspective view of the foot of FIG. 2, with the cutting guide of FIGS. 3A, 3B, 3C, and 3D properly positioned on the first cuneiform and the first metatarsus, and attached to the first cuneiform and the first metatarsus in preparation for resection of the first cuneiform and the first metatarsus, according to one embodiment.

FIG. 6A is a perspective view of the foot of FIG. 2, after resection of the first cuneiform and the first metatarsus, removal of the cutting guide, and placement of the first metatarsus to abut the first cuneiform, according to one embodiment.

FIGS. 6B and 6C are dorsal views of the foot of FIG. 2, before and after correction, respectively, according to one embodiment.

FIGS. 7A, 7B, 7C, and 7D are top perspective, alternative top perspective, front elevation, and bottom perspective views, respectively, of a patient-specific cutting guide according to one alternative embodiment.

FIGS. 8A, 8B, and 8C are dorsal pre-operative, dorsal post-operative, and lateral post-operative views, respectively, of a foot treated with an Evans calcaneal osteotomy, according to one embodiment.

FIGS. 9A and 9B are dorsal post-operative and lateral post-operative views, respectively, of a foot treated with a medializing calcaneal osteotomy, according to one embodiment.

FIG. 10 is a rear, perspective view of the foot of FIG. 2, after performance of an Evans calcaneal osteotomy and a medializing calcaneal osteotomy with patient-specific instruments and/or implants, according to one embodiment.

FIG. 11 is a perspective view of the implant of FIG. 10, in isolation, according to one embodiment.

### DETAILED DESCRIPTION

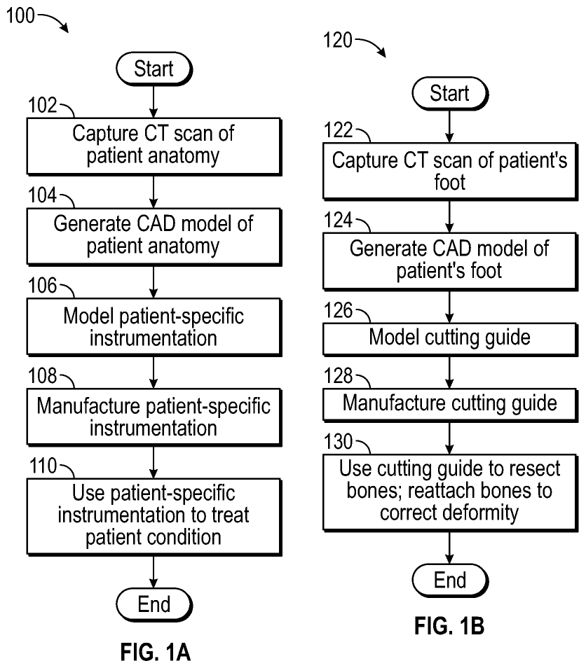

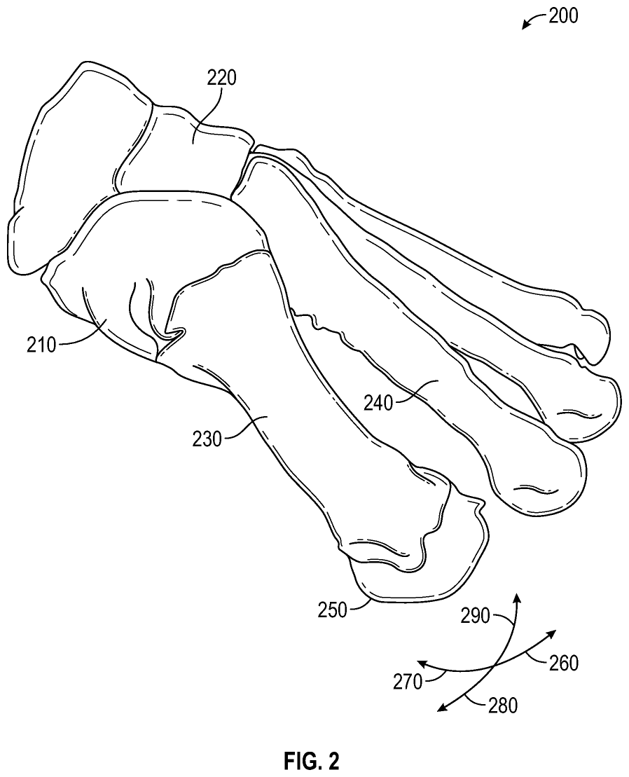

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method, as represented in FIGS. 1A through 1I, is not intended to limit the scope of the disclosure but is merely representative exemplary of exemplary embodiments.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The present disclosure discloses surgical systems and methods by which a bone condition, such as a deformity, may be corrected through the use of patient-specific instrumentation. Known methods of correcting bone conditions are often limited to a finite range of discretely sized instruments. A patient with an unusual condition, or anatomy that falls between instrument sizes, may not be readily treated with such systems. One example is correction of a bunion, in particular, via adjustment of the angulation between a cuneiform and a metatarsus.

FIG. 1A is a flowchart diagram depicting a method 100 for correcting a bone condition, according to one embodiment. The method 100 may be used for any of a wide variety of bone conditions, including but not limited to deformities, fractures, joint failure, and/or the like. Further, the method 100 may provide correction with a wide variety of treatments, including but not limited to arthroplasty, arthrodesis, fracture repair, and/or the like.

As shown, the method 100 may begin with a step 102 in which a CT scan (or another three-dimensional image) of the patient's anatomy is obtained. The step 102 may entail capturing a scan of only the particular bone(s) to be treated, or may entail capture of additional anatomic information, such as the surrounding tissues. Additionally or alternatively, the step 102 may entail receiving a previously captured image, for example, at a design and/or fabrication facility. Performance of the step 102 may result in possession of a three-dimensional model of the patient's anatomy, or three-dimensional surface points that can be used to construct such a three-dimensional model.

After the step 102 has been carried out, the method 100 may proceed to a step 104 in which a CAD model of the patient's anatomy is generated. The CAD model may be of any known format, including but not limited to SolidWorks, Catia, AutoCAD, or DXF. In some embodiments, customized software may be used to generate the CAD model from the CT scan. The CAD model may only include the bone(s) to be treated or may include surrounding tissues. In alternative embodiments, the step 104 may be omitted, as the CT scan may capture data that can directly be used in future steps without the need for conversion.

In a step 106, the CAD model and/or CT scan data may be used to model patient-specific instrumentation that can be used to correct the condition, as it exists in the patient's anatomy. In some embodiments, any known CAD program may be used to view and/or manipulate the CAD model and/or CT scan, and generate one or more instruments that are matched specifically to the size and/or shape of the patient's bone(s). In some embodiments, such instrumentation may include a cutting guide that is attachable to one or more bones, with one or more guide features that facilitate resection of the one or more bones pursuant to a procedure such as arthroplasty or arthrodesis. In some embodiments, performance of the step 106 may include modelling an instrument with a bone apposition surface that is shaped to match the contour of a surface of the bone, such that the bone apposition surface can lie directly on the corresponding contour.

In a step 108, the model(s) may be used to manufacture patient-specific instrumentation and/or implants. This may be done via any known manufacturing method, including casting, forging, milling, additive manufacturing, and/or the like. Additive manufacturing may provide unique benefits, as the model may be directly used to manufacture the necessary instrumentation and/or implants (without the need to generate molds, tool paths, and/or the like beforehand). Such instrumentation may optionally include a cutting guide with the bone apposition surface and one or more guide features as described above.

In addition to or in the alternative to the step 108, the model(s) may be used to select from available sizes of implants and/or instruments and advise the surgeon accordingly. For example, where a range of cutting guides are available for a given procedure, analysis of the CAD data may facilitate pre-operative selection of the optimal cutting guide and/or optimal placement of the cutting guide on the bone. Similarly, if a range of implants may be used for a given procedure, analysis of the CAD data may facilitate pre-operative selection of the optimal implant(s). More particularly, properly-sized spacers, screws, bone plates, and/or other hardware may be pre-operatively selected.

Thus, the result of the step 108 may be provision, to the surgeon, of one or more of the following: (1) one or more patient-specific instruments; (2) one or more patient-specific implants; (3) an instrument, selected from one or more available instrument sizes and/or configurations; (4) an implant, selected from one or more available implant sizes and/or configurations; (5) instructions for which instrument(s) to select from available instrument sizes and/or configurations; (6) instructions for which implant(s) to select from available implant sizes and/or configurations; (7) instructions for proper positioning or anchorage of one or more instruments to be used in the procedure; and (8) instructions for proper positioning or anchorage of one or more implants to be used in the procedure. These items may be provided to the surgeon directly, or to a medical device company or representative, for subsequent delivery to the surgeon.

In a step 110, the manufactured instrumentation may be used in surgery to facilitate treatment of the condition. In some embodiments, this may entail placing the modelled bone apposition surface against the corresponding contour of the bone used to obtain its shape, and then using the guide feature(s) to guide resection of one or more bones. Then the bone(s) may be further treated, for example, by attaching one or more joint replacement implants (in the case of joint arthroplasty), or by attaching bone segments together (in the case of arthrodesis or fracture repair). Prior to completion of the step 110, the instrumentation may be removed from the patient, and the surgical wound may be closed.

As mentioned previously, the method 100 may be used to correct a wide variety of bone conditions. One particular example of the method 100 will be shown and described in connection with FIG. 1B, for correction of a bunion deformity of the foot.

FIG. 1B is a flowchart diagram depicting a method 120 for correcting bunion deformity of the human foot, according to one embodiment. The method 120 may be used to carry out an arthrodesis procedure by which the first metatarsocuneiform joint is removed and the first cuneiform and first metatarsus are secured together in a manner that properly aligns the first metatarsus, providing correction of the deformity.

As shown, the method 120 may begin with a step 122 in which a CT scan (or another three-dimensional image) of the patient's foot is obtained. The step 122 may entail capturing a scan of only the first cuneiform and first metatarsus, or may entail capture of additional anatomic information, such as the entire foot. Additionally or alternatively, the step 122 may entail receipt of previously captured image data. Capture of the entire foot in the step 122 may facilitate proper alignment of the first metatarsus with the rest of the foot (for example, with the second metatarsus). Performance of the step 122 may result in generation of a three-dimensional model of the patient's foot, or three-dimensional surface points that can be used to construct such a three-dimensional model.

After the step 122 has been carried out, the method 120 may proceed to a step 124 in which a CAD model of the relevant portion of the patient's anatomy is generated. The CAD model may optionally include the bones of the entire foot, like the CT scan obtained in the step 122. In alternative embodiments, the step 124 may be omitted in favor of direct utilization of the CT scan data, as described in connection with the step 104.

In a step 126, the CAD model and/or CT scan data may be used to model patient-specific instrumentation that can be used to correct the bunion deformity. Such instrumentation may include a cutting guide that is attachable to the first cuneiform and the first metatarsus, with two guide features that facilitate resection of the cuneiform and the metatarsus in preparation for arthrodesis. In some embodiments, performance of the step 126 may include modelling the cutting guide with a bone apposition surface that is shaped to match contours of the surfaces of the cuneiform and the metatarsus, such that the bone apposition surface can lie directly on the corresponding contours of the first cuneiform and the first metatarsus.

In a step 128, the model(s) may be used to manufacture patient-specific instrumentation and/or instruments. This may include manufacturing the cutting guide with the bone apposition surface and the guide features as described above. As in the step 108, the step 128 may additionally or alternatively involve provision of one or more instruments and/or implants from among a plurality of predetermined configurations or sizes. Further, the step 128 may additionally or alternatively involve provision of instructions for placement and/or anchorage of one or more instruments and/or instruments to carry out the procedure.

In a step 130, the manufactured cutting guide may be used in surgery to facilitate treatment of the condition. Specifically, the bone apposition surface of the cutting guide may be placed against the corresponding contours of the first cuneiform and the first metatarsus. The guide features (for example, slots) may then be positioned on either side of the joint between the first cuneiform and the first metatarsus to guide resection of the first metatarsus and the first cuneiform to remove the intervening joint. The cutting guide may then be removed, and the remaining portions of the first cuneiform and the first metatarsus may be placed to abut each other. The cutting guide may have been shaped such that the cuts made to the first cuneiform and the first metatarsus are properly oriented to bring the first metatarsus back into its proper orientation relative to the rest of the foot. The first cuneiform and the first metatarsus may be secured together through the use of a bone plate or the like. The surgical wound may be closed to allow the foot to heal, and to allow the first cuneiform and the first metatarsus to fuse together.

The method 100 and the method 120 are merely exemplary. Those of skill in the art will recognize that various steps of the method 100 and the method 120 may be reordered, omitted, and/or supplemented with additional steps not specifically shown or described herein.

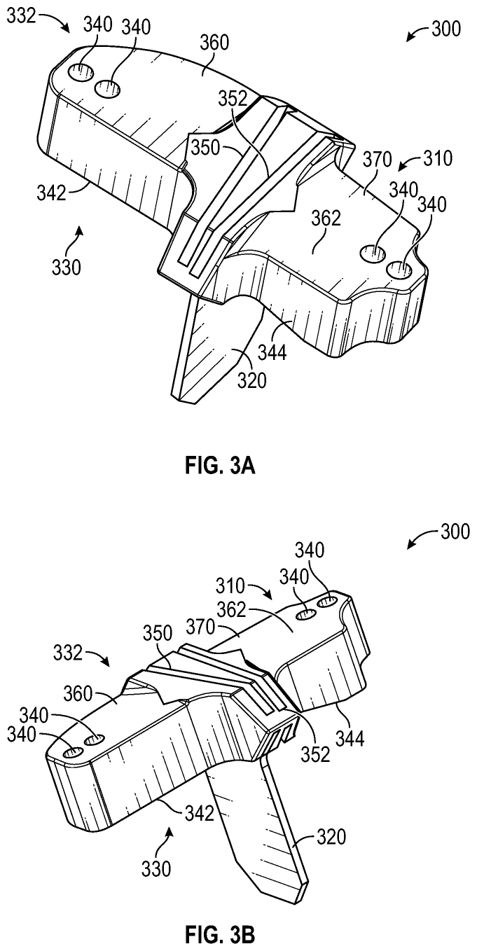

As mentioned previously, the method 120 is only one species of the method 100; the present disclosure encompasses many different procedures, performed with respect to many different bones and/or joints of the body. Exemplary steps and instrumentation for the method 120 will further be shown and described in connection with FIGS. 2 through 7D. Those of skill in the art will recognize that the method 120 may be used in connection with different instruments; likewise, the instruments of FIGS. 2 through 7D may be used in connection with methods different from the method 100 and the method 120.

FIG. 2 is a perspective view of a portion of a foot 200 with a bunion deformity to be treated through use of the method 100 (and more specifically, the method 120) described above. The foot 200 may have a first cuneiform 210, a second cuneiform 220, a first metatarsus 230, and a second metatarsus 240. The first cuneiform 210 and the second cuneiform 220 may be joined together at a first metatarsocuneiform joint, and the first metatarsus 230 and the second metatarsus 240 may be joined together at a second metatarsocuneiform joint.

The first metatarsus 230 may be excessively angled in a medial direction 270 (i.e., toward the lower left-hand corner of the page), causing a painful protrusion at a distal end 250 of the first metatarsus 230, and further causing the phalanges (not shown) attached to the distal end 250 to be angled excessively in a lateral direction 260 (i.e., pointing toward the other phalanges of the foot, rather than pointing directly forward). The excessive medial angulation of the first metatarsus 230 may also result in an excessive gap between the first metatarsus 230 and the second metatarsus 240.

The first metatarsus 230 may further be offset in a plantar direction 280 or in a dorsal direction 290, relative to the remainder of the foot 200. Accordingly, the orientation of the first metatarsus 230 may need to be adjusted to move the distal end 250 in the lateral direction 260 and in the plantar direction 280 and/or in the dorsal direction 290.

Every deformity is different; accordingly, the degree of angular adjustment needed in each direction may be different for every patient. Use of a patient-specific cutting guide may help the surgeon obtain the optimal realignment in the lateral direction 260 and in the plantar direction 280 or the dorsal direction 290. Conversely, use of one of a number of differently-sized cutting guides may provide only approximate correction, as the surgeon may not have a guide that precisely matches the correction needed for the foot 200, and must thus choose the cutting guide that most closely provides the desired correction. Such differently sized cutting guides would not be contoured to fit the first cuneiform 210 or the first metatarsus 230, thus introducing additional potential for error as the surgeon must properly align the selected cutting guide.

Thus, providing a patient-specific cutting guide may provide unique benefits. Specifically, the patient-specific cutting guide may provide precise correction of the deformity present in the foot 200 and may also reduce the likelihood of improper correction due to misalignment of the cutting guide on the foot 200. The optimal cut provided by such a cutting guide may further reduce the likelihood that additional procedures, such as attachment of the first metatarsus 230 to the second metatarsus 240 to each other with screws or the like, will be needed to provide the desired correction. Any such additional procedure carries its own added surgical burden and risk of failure. Thus, the use of patient-specific instrumentation may shorten surgery, accelerate recovery, and reduce the risk of complications.

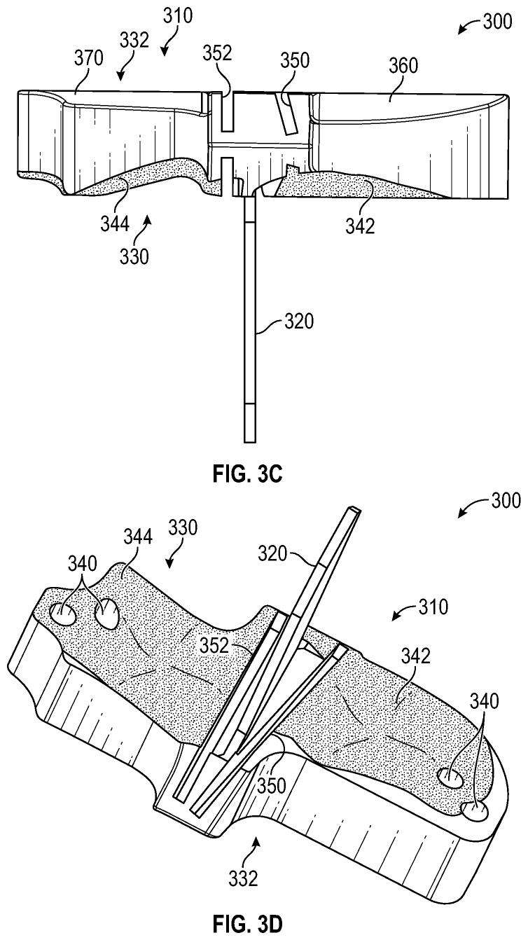

FIGS. 3A, 3B, 3C, and 3D are top perspective, alternative top perspective, front elevation, and bottom perspective views, respectively, of a patient-specific cutting guide, or cutting guide 300, according to one embodiment. The cutting guide 300 may be designed to facilitate resection of the first cuneiform 210 and the first metatarsus 230 with planar cuts at the proper angles to provide dual-plane correction of the orientation of the first metatarsus 230, thereby providing correction in the lateral direction 260 and in the plantar direction 280 or the dorsal direction 290.

As shown, the cutting guide 300 may have a body 310 with a monolithic construction and the general shape of a rectangular prism. The cutting guide 300 may further have a joint alignment feature that helps align the body 310 with the metatarsocuneiform joint between the first cuneiform 210 and the first metatarsus 230. The joint alignment feature may consist of a joint probe 320 that extends from the body 310 and has a blade-like shape. The body 310 may reside on the dorsal surfaces of the first cuneiform 210 and the first metatarsus 230, while the joint probe 320 may protrude into the metatarsocuneiform joint between the first cuneiform 210 and the first metatarsus 230 to provide proper alignment of the body 310 with the metatarsocuneiform joint.

The body 310 may have a bone apposition side 330 that, upon attachment of the body 310 to the first cuneiform 210 and the first metatarsus 230, is to face toward the first cuneiform 210 and the first metatarsus 230. The body 310 may also have an outward-facing side 332 that, upon attachment of the body 310 to the first cuneiform 210 and the first metatarsus 230, faces outward, away from the first cuneiform 210 and the first metatarsus 230. Further, the body 310 may have one or more bone attachment features that facilitate attachment of the body 310 to the first cuneiform 210 and/or the first metatarsus 230. Such bone attachment features may comprise any of a wide variety of holes, spikes, fastening devices, and/or the like. As embodied in FIGS. 3A through 3D, the bone attachment features may take the form of holes 340 that extend from the bone apposition side 330 to the outward-facing side 332. The holes 340 may be shaped to accommodate pins, K-wires, and/or other elongated bone fixation elements that can be anchored in the first cuneiform 210 and/or the first metatarsus 230 to keep the cutting guide 300 in place.

The bone apposition side 330 may be custom contoured to match the shapes of the first cuneiform 210 and/or the first metatarsus 230. As embodied in FIGS. 3A through 3D, the bone apposition side 330 may have a cuneiform apposition portion 342 shaped to lie against the dorsal surface of the first cuneiform 210, and a metatarsus apposition portion 344 shaped to lie against the dorsal surface of the first metatarsus 230. As shown, the cuneiform apposition portion 342 may be contoured to match the contour of the dorsal surface of the first cuneiform 210 on which it is to rest, and the metatarsus apposition portion 344 may similarly be contoured to match the contour of the dorsal surface of the first metatarsus 230 on which it is to rest. Thus, the body 310 may have only one stable position and orientation relative to the first cuneiform 210 and the first metatarsus 230.

Generation of the contours of the cuneiform apposition portion 342 and the metatarsus apposition portion 344 may be performed relative easily in various CAD programs. In some embodiments, the shapes of the corresponding dorsal surfaces of the first cuneiform 210 and the first metatarsus 230 may be obtained directly from the CAD models and/or CT scan data, and simply copied onto the model for the body 310 of the cutting guide 300. Various operations may be used to copy surfaces from one object to another. Additionally or alternatively, various Boolean operations, such as a Boolean subtraction operation, may be used to remove material from a model for the body 310 with a shape that matches the dorsal surfaces of the first cuneiform 210 and the first metatarsus 230.

The body 310 may further have guide features that guide a cutter to resect the first cuneiform 210 and the first metatarsus 230 in the manner needed to make the desired correction. For example, the guide features may be used to guide a planar cutting blade, an arcuate cutting blade, a drill or mill, a burr, and/or the like.

In the embodiment of FIGS. 3A through 3D, the guide features may guide a reciprocating planar blade, such as that of a surgical bone saw, that forms planar cuts in the first cuneiform 210 and the first metatarsus 230. Thus, the guide features may take the form of a first slot 350 and a second slot 352, which may be positioned toward the center of the body 310, on opposite sides of the joint probe 320. Thus, upon proper positioning of the cutting guide 300, the first slot 350 may be positioned over the first cuneiform 210 to facilitate resection of the first cuneiform 210, while the second slot 352 may be positioned over the first metatarsus 230 to facilitate resection of the first metatarsus 230.

In alternative embodiments, a guide feature may be designed to guide a different type cutter, such as a drill, mill, or side-cutting burr. In such embodiments, the guide feature may not be a slot, but may instead be a translatable or rotatable cutter retainer that guides translation and/or rotation of the cutter relative to the bone.

Returning to FIGS. 3A through 3D, the body 310 may further have features that facilitate proper positioning of the cutting guide 300 on the first cuneiform 210 and the first metatarsus 230. More specifically, the body 310 may have a first bone indicator 360 with the text “CUN,” indicating that the end of the body 310 with the first bone indicator 360 is to be positioned over the first cuneiform 210. Similarly, the body 310 may have a second bone indicator 362 with the text “MET,” indicating that the end of the body 310 with the second bone indicator 362 is to be positioned over the first metatarsus 230. In addition, the body 310 may have a side indicator 370 with the text “LEFT,” indicating that the cutting guide 300 is to be used in connection with the patient's left foot. The side indicator 370 may be particularly helpful when bunion corrections are to be provided on both of the patient's feet. In such a case, the surgeon may manufacture or receive two separate cutting guides: one for the left foot (the foot 200 of FIG. 2) and another for the right foot (not shown).

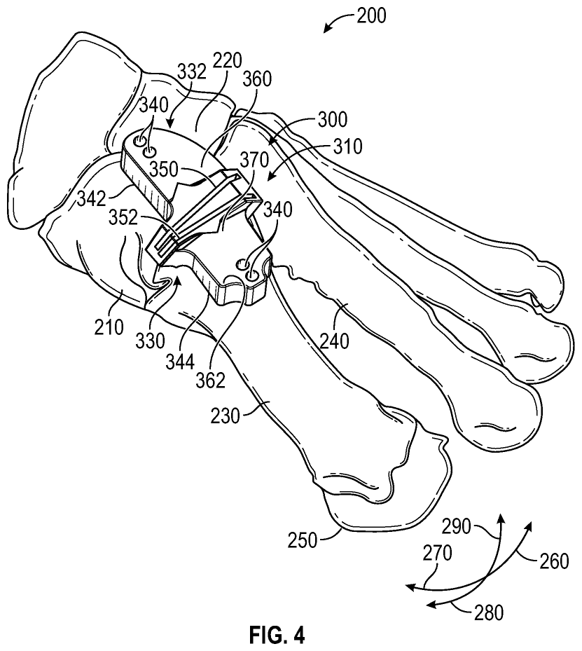

FIG. 4 is a perspective view of the foot 200 of FIG. 2, with the cutting guide 300 of FIGS. 3A, 3B, 3C and 3D properly positioned on the first cuneiform 210 and the first metatarsus 230, but as yet not attached to the first cuneiform 210 and the first metatarsus 230. The surgeon has made the necessary incision(s) to expose the dorsal surfaces of the first cuneiform 210 and the first metatarsus 230, and has inserted the cutting guide 300 such that the cuneiform apposition portion 342 (identified by the first bone indicator 360 on the outward-facing side 332 of the body 310) is resting on the corresponding dorsal surface of the first cuneiform 210, and the metatarsus apposition portion 344 (identified by the second bone indicator 362 on the outward-facing side 332 of the body 310) is resting on the corresponding dorsal surface of the first metatarsus 230. Since the cuneiform apposition portion 342 and the metatarsus apposition portion 344 are contoured to match the bone surfaces on which they rest, the body 310 may readily slide into its proper position on the first cuneiform 210 and the first metatarsus 230.

Notably, the joint probe 320 (not visible) may reside between the first cuneiform 210 and the first metatarsus 230 (i.e., distal to the first cuneiform 210 and proximal to the first metatarsus 230). The surgeon may need to cut the metatarsocuneiform joint between the first cuneiform 210 and the first metatarsus 230 to form a space between the first cuneiform 210 and the first metatarsus 230 to receive the joint probe 320. Positioning the joint probe 320 in this space may further help to ensure that the cutting guide 300 is properly aligned relative to the first cuneiform 210 and the first metatarsus 230.

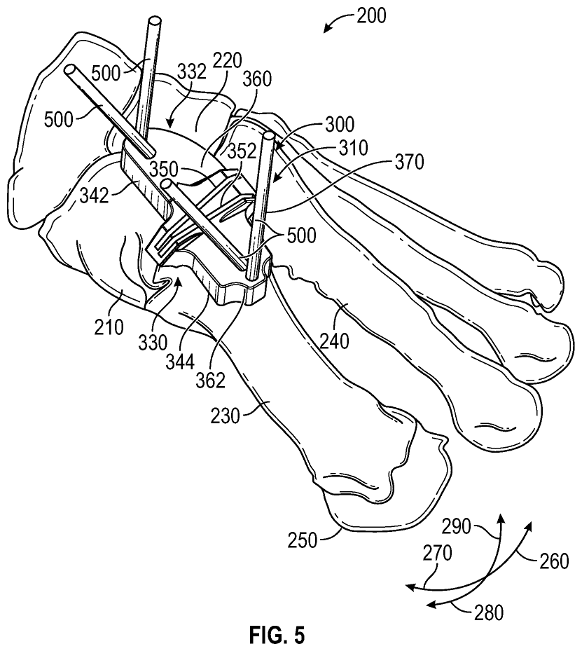

FIG. 5 is a perspective view of the foot 200 of FIG. 2, with the cutting guide 300 of FIGS. 3A, 3B, 3C, and 3D properly positioned on the first cuneiform 210 and the first metatarsus 230, and attached to the first cuneiform 210 and the first metatarsus 230 in preparation for resection of the first cuneiform 210 and the first metatarsus 230. Specifically, pins 500 may be inserted through the holes 340 in the body 310 and anchored in the first cuneiform 210 and the first metatarsus 230. Each of the pins 500 may have a sharp and/or threaded distal end that can penetrate and/or readily be retained in the bone of the first cuneiform 210 or the first metatarsus 230. Additionally or alternatively, a drill or other hole-forming instrument may be used to pre-form holes in the first cuneiform 210 and/or the first metatarsus 230 to receive the distal ends of the pins 500.

As shown, the body 310 may have two holes 340 positioned over the first cuneiform 210, and two holes 340 positioned over the first metatarsus 230. This is merely exemplary; in some embodiments, a cutting guide may be secured to only one of the first cuneiform 210 and the first metatarsus 230, or may be secured to either of the first cuneiform 210 and the first metatarsus 230 with only one pin 500, or with more than two pins 500. Further, in some alternative embodiments, different fasteners may be used, such as screws, clamps, clips, and/or the like.

Once the cutting guide 300 has been secured relative to the first cuneiform 210 and the first metatarsus 230, the first cuneiform 210 and the first metatarsus 230 may be resected. In some embodiments, a reciprocating blade may be inserted into the first slot 350 and moved medially and laterally, between opposite ends of the first slot 350, to make a planar cut that removes the distal end of the first cuneiform 210. Similarly, the reciprocating blade (or a different reciprocating blade) may be inserted into the second slot 352 and moved medially and laterally, between opposite ends of the second slot 352, to make a planar cut that removes the proximal end of the first metatarsus 230. The cuts in the first cuneiform 210 and the first metatarsus 230 may be made in either order. In either case, once both cuts are made, the metatarsocuneiform joint between the first cuneiform 210 and the first metatarsus 230 may be removed, resulting in exposure of “bleeding” bone at the distal end of the first cuneiform 210 and the proximal end of the first metatarsus 230. The cutting guide 300 may be removed, along with some or all of the pins 500. If desired, at least two of the pins 500 may remain in place and used to attach a distractor (not shown) to the first cuneiform 210 and the first metatarsus 230, such that the distractor can temporarily widen the space between the first cuneiform 210 and the first metatarsus 230 to allow for fenestration and/or other preparation of the cut surfaces of the first cuneiform 210 and the first metatarsus 230. Once such preparation has been carried out, the remaining pins 500 may also be removed.

The resulting bleeding and/or prepared bone may readily grow together and fuse, upon abutment of the distal end of the first cuneiform 210 to the proximal end of the first metatarsus 230, particularly with application of some compression across the juncture of the two bones. Since the positions and orientations of the first slot 350 and the second slot 352 were carefully selected to provide the proper correction, the first metatarsus 230 may be positioned to abut the first cuneiform 210, resulting in reorientation of the first metatarsus 230 to a desired orientation, relative to the lateral direction 260 and the plantar direction 280 and/or the dorsal direction 290. Further, the surgeon may optionally rotate the first metatarsus 230, relative to the first cuneiform 210, about an axis perpendicular to the cutting planes, if desired.

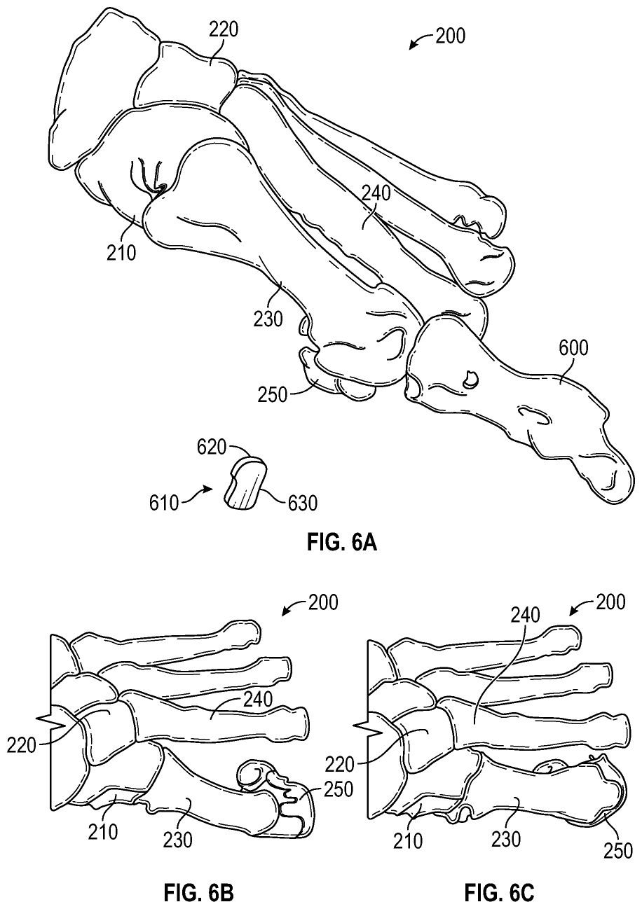

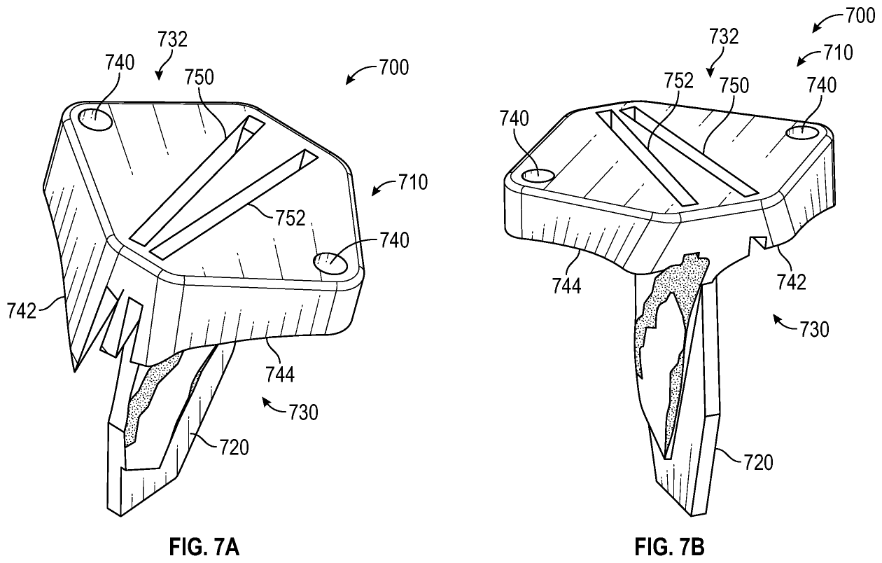

FIG. 6A is a perspective view of the foot 200 of FIG. 2, after resection of the first cuneiform 210 and the first metatarsus 230, removal of the cutting guide 300, and placement of the first metatarsus 230 to abut the first cuneiform 210. As shown, the distal end 250 of the first metatarsus 230 may now be positioned much closer to the second metatarsus 240, in a more natural position. Further, FIG. 6A depicts a first proximal phalanx 600, which may now be properly oriented generally parallel to the other phalanges (not shown), rather than pointing in the lateral direction 260. If desired, further steps may be performed relative to the joint between the first metatarsus 230 and the first proximal phalanx 600 in order to keep them in the proper relative orientation. The distal end 250 may also have been shifted in the plantar direction 280 or in the dorsal direction 290 from the position of FIG. 2. Thus, the desired dual-plane correction of the orientation of the first metatarsus 230 may be complete.

The first metatarsus 230 may be secured to the first cuneiform 210, at least until proper bone in-growth has occurred between the first cuneiform 210 and the first metatarsus 230. In some embodiments, a bone plate (not shown) or other fastener (not shown) may be used to secure the first cuneiform 210 and the first metatarsus 230 together. Additional hardware (not shown) may be used to stabilize the position and/or orientation of the first proximal phalanx 600 relative to the first metatarsus 230, if desired. The surgical wound may be closed, and the foot 200 may be allowed to heal with the bunion deformity corrected.

FIGS. 6B and 6C are dorsal views of the foot 200, before and after correction, respectively. FIGS. 6B and 6C illustrate the correction of the angulation of the first metatarsus 230, by which the distal end 250 of the first metatarsus 230 is moved in the lateral direction 260. In some embodiments, an implant 610 may be inserted in the space between the first metatarsus 230 and the first cuneiform 210 in order hold the first metatarsus 230 and the first cuneiform 210 together and/or facilitate bony fusion between the first metatarsus 230 and the first cuneiform 210.

In some embodiments, the implant 610 may be patient-specific. For example, the implant 610 may have a cuneiform-facing side 620 that is shaped and/or sized to be secured to the adjoining, resected surface of the first cuneiform 210, and a metatarsus-facing side 630 that is shaped and/or sized to be secured to the adjoining, resected surface of the first metatarsus 230. As the resections made to the first metatarsus 230 and the first cuneiform 210 may both planar, the cuneiform-facing side 620 and/or the metatarsus-facing side 630 may also be planar. However, the cuneiform-facing side 620 and/or the metatarsus-facing side 630 may advantageously each be shaped to match the profile of the resected surface of the first cuneiform 210 and the first metatarsus 230, respectively.

This shaping may be accomplished by custom-designing the implant 610 for the patient, using the same models (for example, from CT scans) of the first metatarsus 230 and the first cuneiform 210 that were used to generate the cutting guide 300. Thus, the implant 610 may have a shape that provides secure attachment and/or fusion between the first metatarsus 230 and the first cuneiform 210 while avoiding proud edges or other protruding features that could otherwise interfere with surrounding tissues.

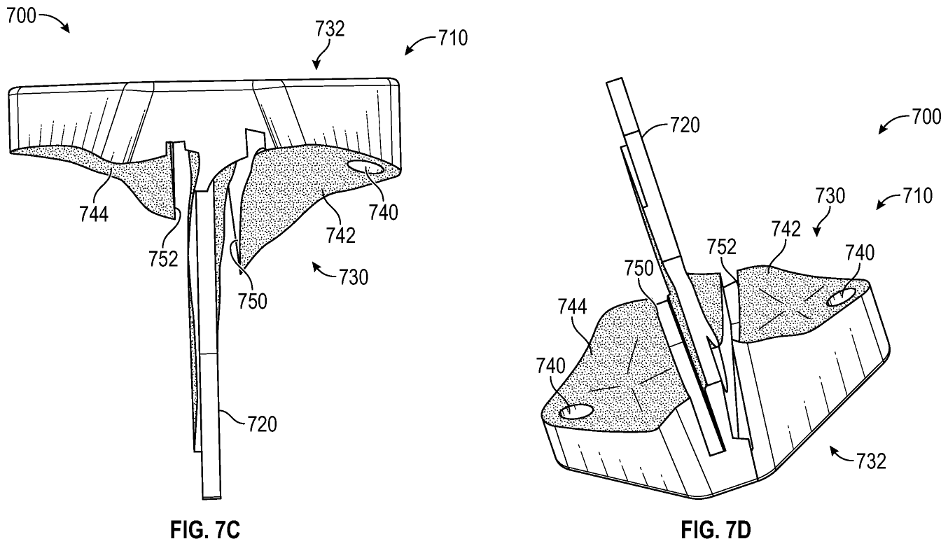

As indicated previously, the cutting guide 300 is only one of many patient-specific instruments that may be used in connection with the method 100 and/or the method 120. An alternative cutting guide suitable for use with the method 120 will be shown and described in connection with FIGS. 7A, 7B, 7C, and 7D.

FIGS. 7A, 7B, 7C, and 7D are top perspective, alternative top perspective, front elevation, and bottom perspective views, respectively, of a patient-specific cutting guide, or cutting guide 700, according to one alternative embodiment. The cutting guide 700 may be used to correct a bunion deformity, such as that of the foot 200 of FIG. 2. Thus, the cutting guide 700 may also be designed to facilitate resection of the first cuneiform 210 and the first metatarsus 230 with planar cuts at the proper angles to provide dual-plane correction of the orientation of the first metatarsus 230, thereby providing correction in the lateral direction 260 and in the plantar direction 280 or the dorsal direction 290.

As shown, the cutting guide 700 may have a body 710 with a monolithic construction and the general shape of a rectangular prism. The cutting guide 700 may further have a joint alignment feature that helps align the body 710 with the metatarsocuneiform joint between the first cuneiform 210 and the first metatarsus 230. The joint alignment feature may consist of a joint probe 720 that extends from the body 710 and has a blade-like shape. The body 710 may reside on the dorsal surfaces of the first cuneiform 210 and the first metatarsus 230, while the joint probe 720 may protrude into the metatarsocuneiform joint between the first cuneiform 210 and the first metatarsus 230 to provide proper alignment of the body 710 with the metatarsocuneiform joint. Notably, the joint probe 720 may have surfaces that are not simply planar, but rather have some contouring by which the shape of the joint probe 720 is matched to the adjoining surfaces of the first cuneiform 210 and/or the first metatarsus 230. Such contouring of the joint probe 720 may enable more precise alignment of the body 710 with the metatarsocuneiform joint.

The body 710 may have a bone apposition side 730 that, upon attachment of the body 710 to the first cuneiform 210 and the first metatarsus 230, is to face toward the first cuneiform 210 and the first metatarsus 230. The body 710 may also have an outward-facing side 732 that, upon attachment of the body 710 to the first cuneiform 210 and the first metatarsus 230, faces outward, away from the first cuneiform 210 and the first metatarsus 230. Further, the body 710 may have one or more bone attachment features that facilitate attachment of the body 710 to the first cuneiform 210 and/or the first metatarsus 230. Such bone attachment features may comprise any of a wide variety of holes, spikes, fastening devices, and/or the like. As embodied in FIGS. 7A through 7D, the bone attachment features may take the form of holes 740 that extend from the bone apposition side 330 to the outward-facing side 332. The holes 340 may be shaped to accommodate pins, K-wires, and/or other elongated bone fixation elements that can be anchored in the first cuneiform 210 and/or the first metatarsus 230 to keep the cutting guide 700 in place. As embodied in FIGS. 7A through 7D, only one hole 340 may be present on each side of the body 710. Thus, the body 710 may be secured to the first cuneiform 210 with only a single pin or K-wire (not shown) and to the first metatarsus 230 with only another single pin or K-wire (not shown).

The bone apposition side 730 may be custom contoured to match the shapes of the first cuneiform 210 and/or the first metatarsus 230. As embodied in FIGS. 7A through 7D, the bone apposition side 730 may have a cuneiform apposition portion 742 shaped to lie against the dorsal surface of the first cuneiform 210, and a metatarsus apposition portion 744 shaped to lie against the dorsal surface of the first metatarsus 230. As shown, the cuneiform apposition portion 742 may be contoured to match the contour of the dorsal surface of the first cuneiform 210 on which it is to rest, and the metatarsus apposition portion 744 may similarly be contoured to match the contour of the dorsal surface of the first metatarsus 230 on which it is to rest. Thus, the body 710 may have only one stable position and orientation relative to the first cuneiform 210 and the first metatarsus 230.

Like the cuneiform apposition portion 342 and the metatarsus apposition portion 344 of the cutting guide 300, generation of the contours of the cuneiform apposition portion 742 and the metatarsus apposition portion 744 may be performed relative easily in various CAD programs through surface copy operations, Boolean operations, and/or the like.

The body 710 may further have guide features that guide a cutter to resect the first cuneiform 210 and the first metatarsus 230 in the manner needed to make the desired correction. For example, the guide features may be used to guide a planar cutting blade, an arcuate cutting blade, a drill or mill, and/or the like.

In the embodiment of FIGS. 7A through 7D, the guide features may guide a reciprocating planar blade, such as that of a surgical bone saw, that forms planar cuts in the first cuneiform 210 and the first metatarsus 230. Thus, the guide features may take the form of a first slot 750 and a second slot 752, which may be positioned toward the center of the body 710, on opposite sides of the joint probe 720. Thus, upon proper positioning of the cutting guide 700, the first slot 750 may be positioned over the first cuneiform 210 to facilitate resection of the first cuneiform 210, while the second slot 752 may be positioned over the first metatarsus 230 to facilitate resection of the first metatarsus 230.

In operation, the cutting guide 700 may be used in a manner similar to that of the cutting guide 300. However, the cutting guide 700 may only be secured to each of the first cuneiform 210 and the first metatarsus 230 with a single pin or K-wire (not shown), as mentioned previously. Further, the cutting guide 700 is smaller than the cutting guide 300. Thus, the cutting guide 700 may be placed through a smaller, less invasive incision. One advantage to patient-specific instrumentation may be that instruments may be made smaller, since they are not limited to certain sizes. Many known instruments come in discrete sizes, each of which is designed to accommodate a range of patient anatomic dimensions. Thus, for given patient anatomy, the instrument must be large enough to treat the anatomy at either end of its range. This typically requires the instrument to be oversized for many anatomic dimensions it is designed to treat. Notably, the cutting guide 700 is merely one compact example; other cutting guides may be made even smaller; in some embodiments, cutting guides may be made that have a smaller width between holes (e.g., holes 740 on the cutting guide 700). As long as the holes are sufficiently far apart to avoid interference of the pins 500 with the operation of the cutting blade, the cutting guide may function appropriately. Thus, Lapidus and other procedures may be accomplished through a very narrow incision through the use of patient-specific instrumentation.

Those of skill in the art will recognize that a wide variety of differently configured cutting guides may be used in conjunction with the method 120 set forth above. Further, a wide variety of patient-specific instruments may be used in connection with the method 100, including but not limited to cutting guides, gages, implant positioning guides, joint distractors, joint compressors, soft tissue retractors, and the like.

Furthermore, patient-specific cutting guides may be used for various other procedures on the foot, or on other bones of the musculoskeletal system. Patient-specific cutting guides may be used for various procedures involving osteotomy, including but not limited to arthroplasty, fusion, and deformity correction procedures. According to one example, patient-specific cutting guides similar to the cutting guide 300 and the cutting guide 700 may be used for the metatarsophalangeal (“MTP”) joint. A method similar to the method 100 may be employed.

In some embodiments, one or more articulating surfaces of a joint may be replaced and/or resurfaced. For example, for the MTP joint, a patient-specific cutting guide may be used to determine the angles of cuts on the distal metatarsus or the proximal phalanx in preparation for replacement or resurfacing of the metatarsal head and/or the proximal phalangeal base. Implants for either the metatarsus or the phalanx may be customized to match the patient's original anatomy, such as the curvature of the MTP joint. In other embodiments, an MTP joint may be fused through the use of patient-specific cutting guides. Patient-specific cutting guides may be used to treat (for example, via fusion, resurfacing, and/or arthroplasty) any joint in the body, using methods similar to the method 100.

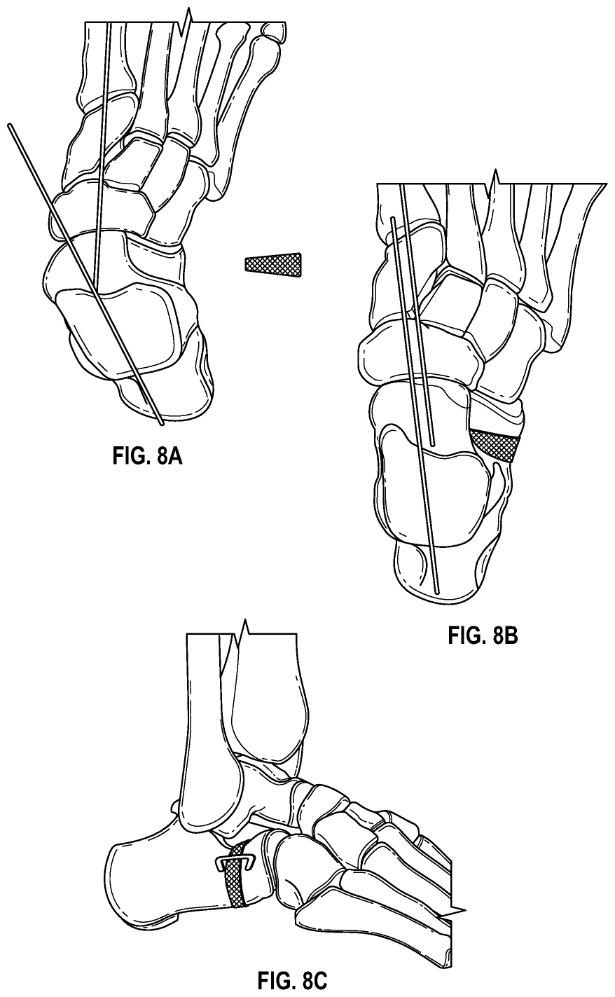

According to other examples, patient-specific cutting guides may be used to carry out an Evans calcaneal osteotomy and/or a medializing calcaneal osteotomy. Patient-specific instruments will be shown and described in connection with FIGS. 8A through 11, in relation to an Evans calcaneal osteotomy, and a medializing calcaneal osteotomy.

FIGS. 8A, 8B, and 8C are dorsal pre-operative, dorsal post-operative, and lateral post-operative views, respectively, of a foot treated with an Evans calcaneal osteotomy, according to one embodiment. Outward rotation of the foot may occur in patients with flatfoot. An Evans or lateral column lengthening procedure is sometimes performed for these patients. An incision is made on the outside of the foot, and the front half of the heel bone is cut. A bone wedge (typically either titanium or a bone-based graft) is then placed into the cut area of the heel bone. This wedge helps to “lengthen” the heel bone and rotate the foot back into its correct position. The wedge is usually kept in place using screws or a surgical staple.

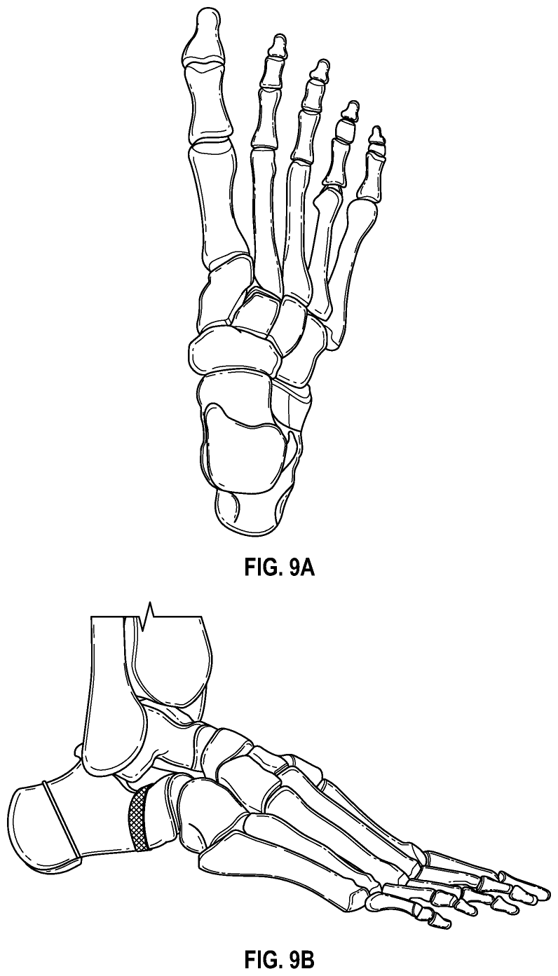

FIGS. 9A and 9B are dorsal post-operative and lateral post-operative views, respectively, of a foot treated with a medializing calcaneal osteotomy, according to one embodiment. A medializing calcaneal osteotomy (heel slide) procedure is often used when the calcaneus (heel bone) has shifted out from underneath the leg. An incision is made on the outside of the heel, and the back half of the heel bone is cut and slid back underneath the leg. The heel is then fixed in place using metal screws or a plate. This also helps to reposition the Achilles tendon towards the center of the ankle/rearfoot. The medializing calcaneal osteotomy can be used in place of, or in addition to, an Evans calcaneal osteotomy.

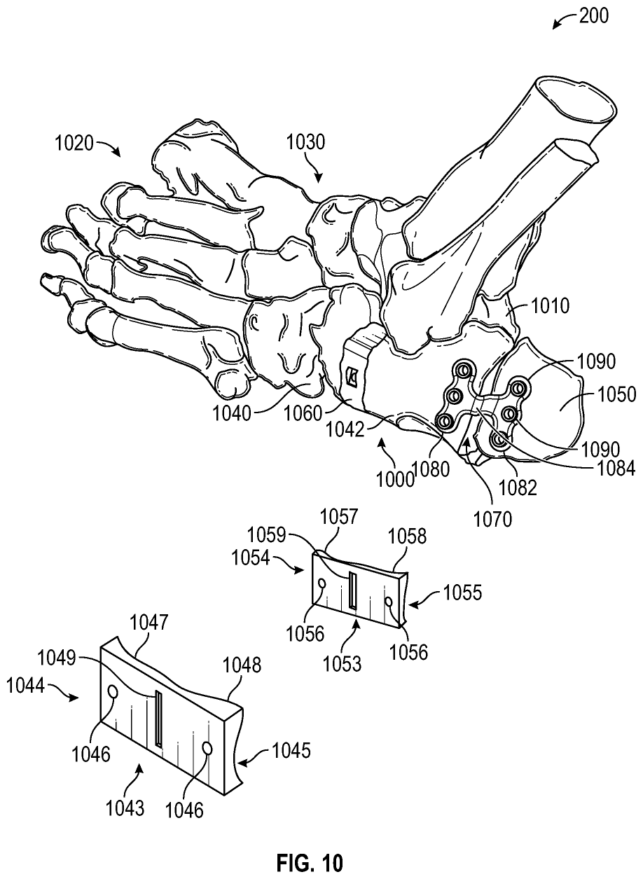

FIG. 10 is a rear, perspective view of the foot 200 of FIG. 2, after performance of an Evans calcaneal osteotomy and a medializing calcaneal osteotomy with patient-specific instruments and/or implants, according to one embodiment. The foot 200 may have a calcaneus 1000 and a talus 1010, in addition to the metacarpals 1020 and cuneiforms 1030 depicted in FIG. 2. Pursuant to the Evans calcaneal osteotomy, an anterior portion of the calcaneus 1000 may be cut along the medial-lateral direction to separate a first bone segment 1040 of the calcaneus 1000 from a second bone segment 1042 of the calcaneus 1000. The second bone segment 1042 may be reoriented medially, relative to the first bone segment 1040, such that a heel 1050 of the calcaneus 1000 is moved medially, simulating a natural, healthy arch in the foot 200.

The cut between the first bone segment 1040 and the second bone segment 1042 may be carried out virtually (for example, in CAD) on a model of the calcaneus 1000 obtained from a CT scan or other imaging of the patient's foot. Thus, the optimal realignment of the posterior end of the calcaneus 1000 can be obtained. If desired, a patient-specific cutting guide, or cutting guide 1043, may be generated in order to facilitate resection of the calcaneus 1000.

As shown, the cutting guide 1043 may have a first end 1044 and a second end 1045, each of which has a bone attachment feature 1046. The bone attachment features 1046 may be used to secure the first end 1044 and the second end 1045 to the first bone segment 1040 and the second bone segment 1042, respectively. The first end 1044 may have a first bone engagement surface 1047 that is shaped to match a corresponding contour on the first bone segment 1040, and the second end 1045 may have a second bone engagement surface 1048 that is shaped to match a corresponding contour on the second bone segment 1042. Thus, the cutting guide 1043 may naturally lie flush with the surface of the calcaneus 1000, in the optimal position on the calcaneus 1000 to facilitate resection of the calcaneus 1000 to divide the first bone segment 1040 from the second bone segment 1042. The cutting guide 1043 may have a guide feature 1049, such as a slot, that can be used to guide a cutter to form a single cut between the first bone segment 1040 and the second bone segment 1042.

After the cut has been made to split the calcaneus 1000 into the first bone segment 1040 and the second bone segment 1042, the surgeon may angle the second bone segment 1042 relative to the first bone segment 1040 in the predetermined (previously modeled) relative orientation. This reorientation between the first bone segment 1040 and the second bone segment 1042 may leave a wedge-shaped gap between the first bone segment 1040 and the second bone segment 1042. In order to maintain the desired relative orientation, an implant 1060 with a wedge shape may be inserted into the gap and secured to the first bone segment 1040 and the second bone segment 1042. The implant 1060 may be fabricated specifically for the patient, since the precise angulation and position of the realignment may also be patient specific. As shown, the implant 1060 may have exterior surfaces that are contoured to match the contours of the adjoining portions of the first bone segment 1040 and the second bone segment 1042. Thus, the implant 1060 may provide secure fixation, while not protrude beyond the adjoining surfaces of the first bone segment 1040 and the second bone segment 1042. Thus, the implant 1060 may be devoid of proud edges or other protrusions that could otherwise interfere with motion between the calcaneus 1000 and the talus 1010, or with surrounding soft tissues, thus interfering with the patient's post-operative gait.

The implant 1060 may be made of any biocompatible material, including but not limited to Titanium and alloys thereof, stainless steel, PEEK, and/or the like. The implant 1060 may be formed by any method known in the art, including but not limited to forging, casting, milling, additive manufacturing, and/or the like. In some embodiments, the implant 1060 may have an interior void that can be filled with bone graft or other material designed to promote boney in-growth between the cut surfaces of the first bone segment 1040 and the second bone segment 1042. In alternative embodiments, the implant 1060 may have a mesh and/or lattice structure that facilitates such boney in-growth, which structure may be formed via additive manufacturing.

As mentioned previously, a medializing calcaneal osteotomy may optionally be performed in addition to or in place of the Evans calcaneal osteotomy. As shown, the heel 1050 may be cut from the remainder of the second bone segment 1042 and may be displaced medially. This displacement may also help to restore normal gait and tendon function in the foot 200, particularly when coupled with the Evans calcaneal osteotomy. The proper displacement of the heel 1050 relative to the remainder of the second bone segment 1042 may be determined based on analysis of the CAD models from scans of the foot 200. If desired, the model of the calcaneus 1000 may be divided and manipulated in CAD to simulate the repositioning of the heel 1050 pursuant to the medializing calcaneal osteotomy. Thus, the alignment of the heel 1050 relative to the remainder of the foot 200 can easily be assessed and optimized prior to surgery.

Such preoperative alignment and planning may be particularly useful where multiple procedures, such as the Evans calcaneal osteotomy and the medializing calcaneal osteotomy, are combined for a single patient. Without such planning, it may be difficult to properly assess the effect of the combined procedures on the patient's anatomy. For example, the effect of the Evans calcaneal osteotomy, and that of the medializing calcaneal osteotomy, is to shift the heel 1050 medially. The combined shift may be difficult to assess in the operating room but may be much more easily and accurately gauged via manipulation of the modeled anatomy.

In some embodiments, one or more additional procedures may be carried out, in addition to or in the alternative to those of FIG. 9. For example, in addition to or in the alternative to the Evans calcaneal osteotomy and the medializing calcaneal osteotomy, a cotton osteotomy and/or a first metatarsal midfoot osteotomy may be performed. Patient-specific cutting guides may be designed, fabricated, and surgically used to facilitate any of these procedures through the presence of bone engagement surfaces that are shaped to rest on the particular bony surfaces adjacent to the osteotomy.

As in the case of the Evans calcaneal osteotomy, a custom cutting guide, or cutting guide 1053, may be generated to help the surgeon obtain the correction that was previously modeled and/or planned using the computer models of the foot 200. The cutting guide 1053 may have a structure and function similar to that of the cutting guide 1043 used for the Evans calcaneal osteotomy. Such a cutting guide may have contoured surfaces that match the contours of the adjoining boney surfaces of the remainder of the second bone segment 1042 and/or the heel 1050.

More specifically, the cutting guide 1053 may have a first end 1054 and a second end 1055, each of which has a bone attachment feature 1056. The bone attachment features 1056 may be used to secure the first end 1054 and the second end 1055 to the second bone segment 1042 and the heel 1050, respectively. The first end 1054 may have a first bone engagement surface 1057 that is shaped to match a corresponding contour on the second bone segment 1042, and the second end 1055 may have a second bone engagement surface 1058 that is shaped to match a corresponding contour on the heel 1050. Thus, the cutting guide 1053 may naturally lie flush with the surface of the calcaneus 1000, in the optimal position on the calcaneus 1000 to facilitate resection of the calcaneus 1000 to divide the second bone segment 1042 from the heel 1050. The cutting guide 1053 may have a guide feature 1059, such as a slot, that can be used to guide a cutter to form a single cut between the second bone segment 1042 and the heel 1050.

In order to maintain the heel 1050 in the proper position relative to the remainder of the second bone segment 1042, a bone plate 1070 may be secured to the heel 1050 and to the remainder of the second bone segment 1042. The bone plate 1070 may include a first end 1080 secured to the remainder of the second bone segment 1042, a second end 1082 secured to the heel 1050, and an intermediate portion 1084 that extends from the first end 1080 to the second end 1082, and provides the desired medial shift between the first end 1080 and the second end 1082. The first end 1080 and the second end 1082 may be secured to the remainder of the second bone segment 1042 and to the heel 1050, respectively, through the use of screws 1090.

Like the implant 1060, the bone plate 1070 may be made of any known biocompatible material, through the use of any manufacturing process known in the art. In some embodiments, the bone plate 1070 may also be fabricated specifically for the foot 200, enabling the bone plate 1070 to maintain precisely the desired level of correction. When made specifically for the foot 200 in combination with each other, the implant 1060 and the bone plate 1070 may provide a highly predictable, precise, and customizable level of correction of the flat foot deformity.

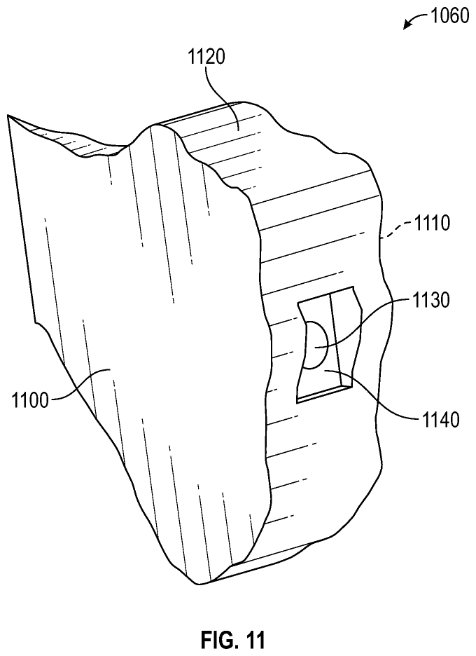

FIG. 11 is a perspective view of the implant 1060, in isolation. As shown, the implant 1060 may have a first bone-facing surface 1100 that is generally flat and shaped to match the cut surface of the first bone segment 1040. The first bone-facing surface 1100 is shown in FIG. 11 with a smooth shape; however, in alternative embodiments, the first bone-facing surface 1100 may be roughened and/or may have teeth, spikes, ridges, and/or other features intended to penetrate the first bone segment 1040 in order to provide for more secure engagement of the implant 1060 with the first bone segment 1040. Similarly, the implant 1060 may have a second bone-facing surface 1110 (not visible) that is also generally flat and shaped to match the cut surface of the second bone segment 1042. Like the first bone-facing surface 1100, the second bone-facing surface 1110 may be roughened or have protruding features that strengthen engagement of the implant 1060 with the second bone segment 1042. If desired, the implant 1060 may be further held in place through the use of bone screws, cement, one or more bone plates, and/or other features known in the art to secure an implant to bone.

The edges of the first bone-facing surface 1100 and the second bone-facing surface 1110 may be shaped to line up with the edges of the cut surfaces of the first bone segment 1040 and the second bone segment 1042, respectively. The implant 1060 may also have a contoured surface 1120 that extends between the edges of the first bone-facing surface 1100 and the second bone-facing surface 1110. The contoured surface 1120 may also be contoured to match the contours of the adjoining portions of the first bone segment 1040 and the second bone segment 1042. Thus, the contoured surface 1120 may provide a continuous surface, devoid of protrusions, that extends between the adjoining surfaces of the first bone segment 1040 and the second bone segment 1042.

A threaded hole 1130 may optionally be provided in the contoured surface 1120. The threaded hole 1130 may be used to secure the implant 1060 to an insertion instrument, a positioning instrument, and/or a removal instrument. The threaded hole 1130 may be formed in a recess 1140 in the contoured surface 1120 so that the threaded hole 1130 can have the desired orientation, without affecting the shape of the contoured surface 1120 more than necessary. Of course, many other features may be used to secure an instrument to the implant 1060, including various clips, clamps, fasteners, and interfacing features, as known in the art.

The present disclosure is not limited to cutting guides or extremity procedures. In some embodiments, patient-specific instrumentation may be used to correct a wide variety of bone conditions. Such conditions include, but are not limited to, any angular deformities from within one bone segment in either the lower or upper extremities (for example, tibial deformities, calcaneal deformities, femoral deformities, and radial deformities). The present disclosure may also be used to treat an interface between two bone segments (for example, the ankle joint, metatarsal cuneiform joint, lisfranc's joint, complex charcot deformity, wrist joint, knee joint, etc.). As one example, an angular deformity or segmental malalignment in the forefoot may be treated, such as is found at the metatarsal cuneiform level, the midfoot level such as the navicular cuneiform junction, hindfoot at the calcaneal cubiod or subtalar joint or at the ankle between the tibia and talar junction. Additionally, patient-specific instruments could be used in the proximal leg between two bone segments or in the upper extremity such as found at the wrist or metacarpal levels.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. A system for correcting a bunion present in one or more bones of a patient's foot, the system comprising:
a cutting guide comprising:
a first bone engagement surface shaped to match a first contour on a first bone of one or more bones of a patient's foot; and
a first guide feature that, with the first bone engagement surface overlying the first contour, is positioned to guide resection of the one or more bones as part of a bunion correction osteotomy;
wherein the first bone is selected from a group consisting of:
a metatarsus; and
a cuneiform.

2. The system of claim 1, wherein at least one of the first bone engagement surface and the first guide feature is defined at least partially based on a bone model of a bone of a patient's foot, the bone model defined based on medical imaging of at least part of the bone of a patient's foot.

3. The system of claim 1, wherein the first bone engagement surface is configured to match a second contour on a second bone of the one or more bones.

4. The system of claim 1, wherein the cutting guide further comprises a second bone engagement surface shaped to match a second contour on a second bone of the one or more bones.

5. The system of claim 4, wherein the cutting guide further comprises a second guide feature that, with the first bone engagement surface overlying the first contour and the second bone engagement surface overlying the second contour, is positioned to guide resection of another one the one or more bones as part of a bunion correction osteotomy.

6. The system of claim 5, wherein the another one the one or more bones is a bone opposite the first bone within a joint of the patient.

7. The system of claim 4, wherein the cutting guide further comprises:
a first end having the first bone engagement surface;
a second end having the second bone engagement surface;
a first bone attachment feature positioned to secure the first end to the first bone; and
a second bone attachment feature positioned to secure the second end to the second bone.

8. The system of claim 7, wherein at least one of the first bone attachment feature and the second bone attachment feature comprises a plurality of holes configured to accept a plurality of fasteners configured to extend through the holes and into bone.

9. The system of claim 4, further comprising a joint alignment feature configured to align the cutting guide with a joint between the first bone and the second bone.

10. The system of claim 1, wherein cutting guide comprises:
a cuneiform apposition portion configured to lie against a dorsal surface of a cuneiform of the patient; and
a metatarsal apposition portion configured to lie against a dorsal surface of a metatarsal of the patient.

11. A cutting guide for correcting a bunion present in a patient's foot, the cutting guide comprising:
a first bone engagement surface shaped to match a first contour on a cuneiform of a patient's foot;
a second bone engagement surface shaped to match a second contour on a metatarsus of the patient's foot;
a first slot that, with the first bone engagement surface overlying the first contour, is positioned to guide resection of the cuneiform to define a first resected surface on the cuneiform;
a second slot that, with the first bone engagement surface overlying the first contour, is positioned to guide resection of the metatarsus to define a second resected surface on the metatarsus; and
wherein the first slot and the second slot are positioned and oriented relative to each other such that, upon joining the first resected surface to the second resected surface and fusing the cuneiform and the metatarsus, the bunion is at least partially corrected.

12. The cutting guide of claim 11, wherein at least one of the first bone engagement surface, the second bone engagement surface, the first slot, and the second slot are defined at least partially based on a bone model of at least one bone of a patient's foot, the bone model defined based on medical imaging of the patient's foot.

13. The cutting guide of claim 12, wherein an orientation of one of the first slot and the second slot is defined at least partially based on the bone model and a desired correction.

14. The cutting guide of claim 11, further comprising:
a first end having the first bone engagement surface;
a second end having the second bone engagement surface;
a first bone attachment feature positioned to secure the first end to the cuneiform; and
a second bone attachment feature positioned to secure the second end to the metatarsus.

15. The cutting guide of claim 14, wherein at least one of the first bone attachment feature and the second bone attachment feature comprises a plurality of holes configured to accept a plurality of fasteners configured to extend through the holes and into bone.

16. The cutting guide of claim 11, further comprising:
a body having a bone apposition side and an outward facing side; and
a joint probe that extends from the bone apposition side, the joint probe configured to align the body with a metatarsocuneiform joint between the cuneiform and the metatarsus.

17. A cutting guide for correcting a bunion present in a patient's foot, the cutting guide comprising:
a bone apposition side comprising:
a cuneiform apposition portion shaped to lie against a first cuneiform;
a metatarsus apposition portion shaped to lie against a first metatarsus;
a first slot positioned to guide resection of the first cuneiform to define a first resected surface on the first cuneiform;
a second slot positioned to guide resection of the first metatarsus to define a second resected surface on the first metatarsus; and
wherein the first slot and the second slot are positioned within the cutting guide and oriented relative to each other such that fusion of the first cuneiform and the first metatarsus by abutting the first resected surface and the second resected surface at least partially corrects the bunion.

18. The cutting guide of claim 17, wherein at least one of the cuneiform apposition portion, the metatarsus apposition portion, the first slot, and the second slot is defined at least partially based on a bone model of one or more bones of a patient's foot, the bone model defined based on medical imaging of the patient's foot.

19. The cutting guide of claim 17, wherein the cuneiform apposition portion is contoured to the contour of a dorsal surface of the first cuneiform and the metatarsus apposition portion is contoured to the contour of a dorsal surface of the first metatarsus.

20. The cutting guide of claim 17, further comprising:
a body comprising the bone apposition side and an outward facing side opposite the bone apposition side; and
a joint probe having a blade shape that extends from the bone apposition side and in to a metatarsocuneiform joint between the first cuneiform and the first metatarsus.
